(19) United States
(12) Patent Application Publication
(10) Pub. No.: US 2018/0228818 A1
(43) Pub. Date: Aug. 16, 2018

(54) PHARMACEUTICAL COMPOSITION COMPRISING ASPRIN, METFORMIN, AND SEROTONIN WITH NON-IONIC SURFACTANT

(71) Applicants: Chien-Hung CHEN, Forest Hills, NY (US); ALS MOUNTAIN LLC, Forest Hills, NY (US)

(72) Inventor: Chien-Hung CHEN, Forest Hills, NY (US)

(21) Appl. No.: 15/516,152

(22) PCT Filed: Oct. 1, 2015
(86) PCT No.: PCT/US2015/053475

§ 371 Date: Mar. 31, 2017

Related U.S. Application Data
(60) Provisional application No. 62/058,150, filed on Oct. 1, 2014.

(51) Int. Cl. A61K 31/616; A61K 31/155; A61K 31/4045; A61K 47/34; A61K 47/10

(52) U.S. Cl. CPC A61K 31/616; A61K 31/155; A61K 47/10; A61K 47/34; A61K 31/4045

(57) ABSTRACT

The present invention is based on the unexpected discovery that a combination of certain known drugs exhibits synergistic effects in treating metabolic syndrome and various other diseases. In particular, the invention comprises a pharmaceutical composition comprising: (1) a therapeutically effective quantity of a first agent that is Metformin or a salt thereof; (2) a therapeutically effective quantity of a second agent that is Aspirin; (3) a therapeutically effective quantity of a third agent that serotonin creatinine sulfate complex; (4) a non-ionic surfactant; and (5) a solvent that is a lower alkanol. A preferred composition comprises metformin hydrochloride, aspirin, and serotonin creatinine sulfate complex for the first, second, and third agents. The invention further comprises methods for the use of these compositions for the treatment of metabolic syndrome, hyperproliferative diseases including cancer, and other diseases and conditions.

### CROSS-REFERENCES TO RELATED APPLICATIONS

This application claims the benefit of U.S. Provisional Patent Application Ser. No. 62/058,150 by Chien-Hung Chen, entitled “Pharmaceutical Composition Comprising Aspirin, Metformin, and Serotonin with Non-Ionic Surfactant,” and filed on Oct. 1, 2014, the contents of which are incorporated herein by this reference.

### FIELD OF THE INVENTION

This invention is directed to a pharmaceutical formulation comprising a first agent, second agent, third agent, solvent and at least a non-ionic surfactant.

### BACKGROUND OF THE INVENTION

Metabolic syndrome is characterized by a group of metabolic risk factors, including abdominal obesity, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels). Metabolic syndrome has become increasingly common in the United States. It is estimated that over 50 million Americans have this disorder. There is a need to develop novel drugs to effectively treat this disorder.

According to the World Health Organization, about five million people die from cancer every year. Drug treatment is one of the three major therapies for cancer. At present, drugs are used to treat cancers by the following mechanisms: interfering with or inhibiting cell division, regulating cell generation cycle, promoting tumor cell apoptosis, inhibiting angiogenesis, inhibiting oncogene activity, promoting tumor-suppressing gene activity, acting as tumor antigens, inhibiting telomerase activities, and interfering with information transfer of tumor cells.

In view of the high mortality rates associated with abnormal proliferative diseases including cancer, there exists a need for an effective treatment for these diseases.

Acquired immunodeficiency syndrome (AIDS), a consequence of infection with the HIV-1 retrovirus, affects over 30 million people worldwide. AIDS is characterized by a number of otherwise very rare opportunistic infections such as Kaposi's sarcoma, caused by the Kaposi's sarcoma-associated herpes virus, Pneumocystis jirovecii pneumonia, and other malignancies and infectious diseases. Patients with AIDS also suffer from severe weight loss, night sweats, swollen lymph nodes, and other consequences of a compromised immune system. In AIDS, CD4⁺T cells are attacked by the virus and greatly reduced in number. Although treatments for AIDS do exist, including treatment with a “cocktail” of three drugs belonging to at least two classes of antiretroviral drugs, such as, for example, two nucleoside analogue reverse transcriptase inhibitors plus either a protease inhibitor or a non-nucleoside reverse transcriptase inhibitor. Although this approach has proved reasonably successful in inhibiting the growth of HIV-1 and preventing the occurrence of opportunistic infections and other symptoms of AIDS, it is not a cure and the effectiveness of drug therapy can be limited by drug resistance, drug toxicity, and possible patient non-compliance. Therefore, there is a need for an improved therapy for AIDS.

### BRIEF SUMMARY OF THE INVENTION

The present invention provides pharmaceutical compositions and methods that are suitable for treating a number of diseases and conditions, including: metabolic syndrome and diseases and conditions associated with metabolic syndrome, including diabetes, obesity, and hypertension; hyperproliferative diseases and conditions including cancer; AIDS; Parkinson's disease; polycystic ovarian syndrome, Alzheimer's disease; osteoporosis; sleep apnea; erectile dysfunction; McArdle disease; and carbohydrate metabolism disorders, as well as being useful for treating aging or fatigue.

This invention is based on the unexpected discovery that a combination of certain known drugs exhibits synergistic effects in treating metabolic syndrome and various other diseases.

In general, a pharmaceutical formulation according to the present invention comprises:

(1) a therapeutically effective quantity of a first agent that is Metformin or a salt thereof;

(2) a therapeutically effective quantity of a second agent that is Aspirin or a salt thereof;

(3) a therapeutically effective quantity of a third agent that is Serotonin Creatinine Sulfate complex;

(4) a component selected from the group consisting of: (i) at least one non-ionic surfactant; and (ii) chitosan or a derivative thereof; and

(5) a solvent.

Preferably, the first agent is metformin or a salt thereof, such as metformin hydrochloride.

Typically, the second agent is selected from the group consisting of aspirin (acetylsalicylic acid) and salts and hydrates thereof

When the composition comprises at least one non-ionic surfactant, typically the at least one non-ionic surfactant is selected from the group consisting of: polyoxyethylene glycol alkyl ethers; polyoxypropylene glycol alkyl ethers; glucoside alkyl ethers; polyoxyethylene glycol octylphenol ethers; polyoxyethyleneglycol alkylphenol ethers (nonoxynols); glycerol alkyl esters; polyoxyethylene glycol sorbitan alkyl esters; cocamides; dodecyldimethylamine oxide; α-hydro-ω-hydroxypoly(oxyethylene)ₐ-poly(oxypropylene)_{b}-poly(oxyethylene)ₐ block copolymers (poloxamers); and polyethoxylated tallow amine. Particularly preferred non-ionic surfactants include poloxamers; among poloxamers, it is more particularly preferred to use both poloxamer 188 and poloxamer 407.

Typically, the at least one non-ionic surfactant acts as one or more of: (1) an absorption enhancer; (2) an emulsifier; (3) a solubilizer; (4) a stabilizer; (5) an agent that controls the physical state of the composition with respect to temperature, in particular, an agent that causes the composition to assume a liquid state at low temperature while causing the composition to assume a gelated solid state at body temperature of 37° C.; or (6) a slow-release agent. When the at least one non-ionic surfactant acts as an absorption enhancer included in the pharmaceutical formulation, it functions to improve the absorption of the pharmacologically active drug. When the at least one non-ionic surfactant acts as an emulsifier included in the pharmaceutical formulation, it functions as a stabilizer for emulsions and preventing the pharmaceutical solutions from separating. The non-ionic surfactant could also function as a solubilizer to enhance the solubility of the pharmaceutical substances. Also, it may act as a stabilizer to stabilize the chemical reaction between different compounds or as a slow-release agent which is capable of gradual release of the active ingredients over a period of time.

In another alternative, the composition can comprise chitosan or a derivative thereof in place of the at least one non-ionic surfactant.

When the second agent is aspirin, the solvent is typically selected from the group consisting of: a lower alkanol selected from the group consisting of ethanol and isopropanol; glycerol; dimethyl sulfoxide (DMSO); and vegetable oil.

Preferably, when the second agent is aspirin, the solvent is a lower alkanol selected from the group consisting of ethanol and isopropanol. More preferably, the solvent is ethanol.

The composition can further comprise at least one stabilizer or excipient; in one alternative, the at least one stabilizer or excipient are sodium metabisulfite and tartaric acid.

The composition can be formulated as an injectable formulation or as a formulation for oral administration.

In one preferred alternative, the composition comprises metformin hydrochloride, serotonin creatinine sulfate, poloxamer 407, poloxamer 188, sodium metabisulfite, aspirin, tartaric acid, ethanol, and water.

The composition can consist essentially of metformin, aspirin, serotonin creatinine sulfate complex, the component selected from the group consisting of: (i) at least one non-ionic surfactant; and (ii) chitosan or a derivative thereof, and the solvent.

The composition can further comprise a pharmaceutically acceptable carrier.

In the composition, the first agent, the second agent, and the third agent, or a combination of more than one of the first agent, the second agent, and the third agent, can be associated with one or more carrier substances to deliver the first agent, the second agent, the third agent, or the combination of more than one of the first agent, the second agent, and the third agent to their intended site or sites of action.

Typically, the composition comprises from about 5 mg to 5000 mg of the first agent, from about 5 mg to about 5000 mg of the second agent, and about 0.1 mg to about 1000 mg of the third agent per unit dose.

Another aspect of the present invention is a method of treating a disease or condition comprising the step of administering a therapeutically effective quantity of a pharmaceutical composition according to the present invention as described above to a subject that has the disease or condition or that is at risk of developing the disease or condition, in order to treat or prevent the occurrence of the disease or condition, wherein the disease or condition is selected from the group consisting of metabolic syndrome, diabetes, obesity, hypertension, cancer, AIDS, Parkinson's disease, polycystic ovarian syndrome, Alzheimer's disease, osteoporosis, sleep apnea, erectile dysfunction, McArdle disease, and a carbohydrate metabolism disorder. Typically, the disease or condition is selected from the group consisting of metabolic syndrome, diabetes, obesity, and hypertension. In another alternative, the disease or condition is cancer. In still another alternative, the disease or condition is selected from the group consisting of Parkinson's disease, polycystic ovarian syndrome, Alzheimer's disease, osteoporosis, sleep apnea, erectile dysfunction, McArdle disease, and a carbohydrate metabolism disorder. The pharmaceutical composition can be administered orally or parenterally.

### DETAILED DESCRIPTION OF THE INVENTION

This invention is based on the unexpected discovery that a combination of certain known drugs exhibits synergistic effects in treating metabolic syndrome and various other diseases. In addition to metabolic syndrome and diseases and conditions associated with metabolic syndrome, the combination of these known drugs can be used to treat hyperproliferative disease (including cancer), AIDS, Parkinson's disease, polycystic ovarian syndrome, Alzheimer's disease, osteoporosis, sleep apnea, erectile dysfunction, McArdle disease, and carbohydrate metabolism disorders. The combination of these known drugs can also be used to treat aging or fatigue. The combination of these known drugs can also be used to treat a disease or condition such as: (1) cardiac dysrhythmias; (2) endometriosis, uterine fibroid (uterine leiomyomata) menorrhagia, cervical erosion, cervical polyp, and related conditions; and (3) defects or disorders of intervertebral discs.

In one aspect, the invention comprises a pharmaceutical formulation comprising:

(1) a therapeutically effective quantity of a first agent that is Metformin or a salt thereof;

(2) a therapeutically effective quantity of a second agent that is Aspirin or a salt thereof;

(3) a therapeutically effective quantity of a third agent that is Serotonin creatinine sulfate complex; and

(4) a component selected from the group consisting of: (i) at least one non-ionic surfactant; and (ii) chitosan or a derivative thereof; and

(5) a solvent.

Preferably, the first agent is metformin or a salt thereof, such as metformin hydrochloride.

Particularly preferred second agent include aspirin (acetylsalicylic acid) and salts and hydrates thereof.

A particularly preferred serotoneric agent is serotonin, typically in the form of serotonin creatinine sulfate complex.

Suitable non-ionic surfactants include, but are not limited to: polyoxyethylene glycol alkyl ethers, including octaethylene glycol monododecyl ether and pentaethylene glycol monodecyl ether; polyoxypropylene glycol alkyl ethers; glucoside alkyl ethers, including decyl glucoside, lauryl glucoside, and octyl glucoside; polyoxyethylene glycol octylphenol ethers such as Triton X-100®; polyoxyethyleneglycol alkylphenol ethers (nonoxynols) such as nonoxynol-9; glycerol alkyl esters such as glyceryl laurate; polyoxyethylene glycol sorbitan alkyl esters; cocamides such as cocamide MEA and cocamide DEA; dodecyldimethylamine oxide; α-hydro-ω-hydroxypoly(oxyethylene)ₐ-poly(oxypropylene)_{b}-poly(oxyethylene)_{b} block copolymers (poloxamers); and polyethoxylated tallow amine.

Other non-ionic surfactants are known in the art.

Preferred non-ionic surfactants are α-hydro-ω-hydroxypoly(oxyethylene)ₐ-poly(oxypropylene)_{b}-poly(oxyethylene)ₐ block copolymers (poloxamers). Particularly preferred non-ionic surfactants are poloxamer 407 and poloxamer 188. The use of one or more poloxamers is disclosed in U.S. Pat. No. 8,481,078 to Holm et al.; U.S. Pat. No. 8,551,524 to Sesha; U.S. Pat. No. 8,604,085 to Turchetta et al.; U.S. Pat. No. 8,747,872 to Baker et al.; and U.S. Pat. No. 8,802,075 to Cooper et al., all of which are incorporated herein by this reference.

As an alternative to one or more non-ionic surfactants, chitosan or a derivative thereof can be used. Preferably, chitosan is used. Chitosan can replace the combination of poloxamer 407 and poloxamer 188 in the preparation of a temperature-sensitive gel that is liquid at low temperature but is in a gelated form at body temperature of 37° C.

The non-ionic surfactants can act as one or more of: (1) an absorption enhancer; (2) an emulsifier; (3) a solubilizer; (4) a stabilizer; (5) an agent that controls the physical state of the composition with respect to temperature, in particular, an agent that causes the composition to assume a liquid state at low temperature while causing the composition to assume a gelated solid state at body temperature of 37° C.; or (6) a slow-release agent.

At low temperature, the non-ionic surfactant solution containing the drug to be released is a fluid solution that can easily be injected intramuscularly into the body via a syringe. At a higher temperature (above the transition temperature at body temperature), the formulation becomes a gel and drug release can be significantly prolonged. In particular, the properties and activity of poloxamer 407 are described in G. Dumortier et al., “A Review of Poloxamer 407 Pharmaceutical and Pharmacological Characteristics,” Pharm. Res. 23: 2709-2728 (2006), incorporated herein by this reference.

A pharmaceutical composition according to the present invention further comprises a solvent. Suitable solvents can be selected by one of ordinary skill in the art depending on the particular first agents, second agents, and third agents included in the composition, and their physical and chemical properties, such as molecular weight, solubility, and relative degree of hydrophobicity or hydrophilicity. When the second agent is aspirin, suitable solvents include, but are not limited to: a lower alkanol selected from the group consisting of ethanol and isopropanol; glycerol; dimethyl sulfoxide (DMSO); and vegetable oil. Typically, when the second agent is aspirin, the solvent is a lower alkanol selected from the group consisting of ethanol and isopropanol. Preferably, when the second agent is aspirin, the solvent is ethanol.

In some alternatives, the composition can further comprise stabilizers or excipients. Suitable stabilizers or excipients include, but are not limited to, sodium metabisulfite and tartaric acid.

The composition can further comprise a pharmaceutically acceptable carrier. Suitable pharmaceutically acceptable carriers are described below.

The composition can be formulated as an injectable formulation. In another alternative, the composition can be formulated as a formulation for oral administration.

In one alternative, the composition consists essentially of the first, second, and third agents, the non-ionic surfactant, and the lower alkanol solvent if present, or, if a pharmaceutically acceptable carrier is included, of these components and a pharmaceutically acceptable carrier. In this alternative, the composition is limited to the specified materials and those that do not materially affect the basic and novel characteristics of the composition.

In one alternative, the first agent is associated with a carrier substance to facilitate the transport of the first agent to an intended site of action of the first agent. The carrier substance can be, but is not limited to, an antibody, an antibody fragment, or a receptor. The first agent can be covalently or noncovalently bound to the carrier substance.

In another alternative, the second agent is associated with a carrier substance to facilitate the transport of the second agent to an intended site of action of the second agent. The carrier substance can be, but is not limited to, an antibody, an antibody fragment, or a receptor. The second agent can be covalently or noncovalently bound to the carrier substance.

In another alternative, the third agent is associated with a carrier substance to facilitate the transport of the third agent to an intended site of action of the third agent. The carrier substance can be, but is not limited to, an antibody, an antibody fragment, or a receptor. The third agent can be covalently or noncovalently bound to the carrier substance.

In yet another alternative, the first agent and the second agent, the first agent and the third agent, the second agent and the third agent, and the first agent, the second agent, and the third agent are each associated with a carrier substance to facilitate the transport of the respective agents to their intended site of action. Each agent (the first agent, the second agent, and the third agent) can be associated with its own carrier substance. Alternatively, two or three of the first agent, the second agent, and the third agent can be bound to the same carrier substance; all possible combinations of binding are within the scope of the invention. If all of the first agent, the second agent, and the third agent are bound to a carrier substance or carrier substances, one, two, or three carrier substances can be used in any possible combination. The first agent, the second agent, and the third agent can be covalently or noncovalently bound to the carrier substance or carrier substances.

Methods for binding the first agent, the second agent, or the third agent to an individual carrier substance are known in the art. Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ϵ-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ϵ-amino group of the side chain of lysine. Many other electrophilic reagents are known that will react with the ϵ-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G.T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that the protein has an amino acid residue with an electrophilic group that is reactive with a nucleophile and the targeting molecule includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail. Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. Such cross-linking reagents and reactions are described in G. T. Hermanson, “Bioconjugate Techniques” (Academic Press, San Diego, 1996), incorporated herein by this reference.

The individual carrier substances can be, but are not limited to, antibodies, hormones, receptor agonists or antagonists, or receptors. As used herein, unless further defined or limited, the term “antibody” encompasses both polyclonal and monoclonal antibodies, as well as genetically engineered antibodies such as chimeric or humanized antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. Receptors are well known in the art and include G-protein coupled receptors (GPCRs). G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, β₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors. Agonists and antagonists specifically binding these receptors can be used as individual carrier substances; suitable receptors, agonists, or antagonists can be selected based on their specificity and the location of the receptors in particular cells or tissues.

Typically, the composition comprises from about 5 mg to 5000 mg of the first agent, from about 5 mg to about 5000 mg of the second agent, and about 0.1 mg to about 1000 mg of the third agent per unit dose. Preferably, the composition comprises from about 5 mg to 5000 mg of metformin hydrochloride, from about 5 mg to about 5000 mg of aspirin, and from about 0.1 mg to about 1000 mg of serotonin creatinine sulfate complex per unit dose.

One alternative of a composition according to the present invention comprises equal volumes of Mixture A and Mixture B, below.

In this alternative, Mixture A comprises, per each 1-mL aliquot: from about 67.5 mg to about 82.5 mg of metformin hydrochloride; from about 4.5 mg to about 5.5 mg of serotonin creatinine sulfate complex; from about 61.875 mg to about 75.625 mg of poloxamer 407; from about 16.875 mg to about 20.625 mg of poloxamer 188; from about 0.45 mg to about 0.55 mg of sodium metabisulfite; and water for injection to a total volume of 1 mL. In this alternative, Mixture B comprises, per each 1-mL aliquot: from about 180 mg to about 220 mg of aspirin; from about 405 mg to about 495 mg of poloxamer 407; from about 4.5 mg to about 5.5 mg of tartaric acid; and anhydrous alcohol to a total volume of 1 mL.

Preferably, Mixture A comprises, per each 1-mL aliquot: 75 mg of metformin hydrochloride; 5 mg of serotonin creatinine sulfate complex; 68.75 mg of poloxamer 407; 18.75 mg of poloxamer 188; 0.5 mg sodium metabisulfite; and water for injection to a total volume of 1 mL. Preferably, Mixture B comprises, per each 1-mL aliquot: 200 mg of aspirin, 450 mg of poloxamer 407; 5 mg of tartaric acid; and anhydrous alcohol to a total volume of 1 mL.

Another aspect of the present invention is a method of treating a disease or condition comprising the step of administering a therapeutically effective quantity of a pharmaceutical composition according to the present invention as described above to a subject that has the disease or condition or that is at risk of developing the disease or condition, in order to treat or prevent the occurrence of the disease or condition, wherein the disease or condition is selected from the group consisting of metabolic syndrome, diabetes, obesity, hypertension, cancer, AIDS, Parkinson's disease, polycystic ovarian syndrome, Alzheimer's disease, osteoporosis, sleep apnea, erectile dysfunction, McArdle disease, and a carbohydrate metabolism disorder. Typically, the disease or condition is selected from the group consisting of metabolic syndrome, diabetes, obesity, and hypertension. In another alternative, the disease or condition is cancer. In still another alternative, the disease or condition is selected from the group consisting of Parkinson's disease, polycystic ovarian syndrome, Alzheimer's disease, osteoporosis, sleep apnea, erectile dysfunction, McArdle disease, and a carbohydrate metabolism disorder.

The pharmaceutical composition can be administered orally or parenterally. Parenteral administration includes, but is not limited to, a route of administration selected from the group consisting of subcutaneous, intracutaneous, intravenous, intramuscular, intraarticular, intraarterial, intrasynovial, intrasternal, intrathecal, intralesional, and intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition for topical administration can be prepared in form of an ointment, a gel, a plaster, an emulsion, a lotion, a foam, a cream of a mixed phase or amphiphilic emulsion system (oil/water-water/oil mixed phase), a liposome, a transfersome, a paste, or a powder.

Any of the compositions described above can also be administered in the form of suppositories for rectal administration. It also can be designed such that the composition is released in the intestine. For example, the composition is confined in a solid sub-unit or a capsule compartment that have respectively a matrix or a wall or a closure comprising an enteric polymer which dissolves or disperses at the pH of the small or large intestine to release the drug substance in the intestine. Suitable such polymers have been described above, for example with reference to U.S. Pat. No. 5,705,189.

The carrier in the pharmaceutical composition must be “acceptable” in the sense that it is compatible with the active ingredients of the composition (and preferably, capable of stabilizing the active ingredients) and not deleterious to the subject to be treated. One or more solubilizing agents can be utilized as pharmaceutical excipients for delivery of an active thiophene compound. Examples of other carriers include colloidal silicon oxide, magnesium stearate, cellulose, sodium lauryl sulfate, and D&C Yellow # 10.

The compositions described above can be used to treat diseases and conditions such as metabolic syndrome, Parkinson's disease, or polycystic ovarian syndrome. The diseases mentioned above also include their associated disorders. For example, disorders associated with metabolic syndrome include atherosclerosis, coronary heart disease, stroke, obesity, diabetes, atherogenic dyslipidemia (e.g., high triglyceride levels, low HDL cholesterol levels, and high LDL cholesterol levels), hypertension, insulin resistance, prothrombotic state (e.g., high fibrinogen or plasminogen activator inhibitor-1 levels), and proinflammatory state (e.g., elevated C-reactive protein levels).

The compositions described above can also be used to treat additional diseases and conditions, including hyperproliferative diseases and Alzheimer's disease. Hyperproliferative diseases include benign tumors and malignant tumors, as well as non-tumor hyperproliferative diseases. Benign tumors include, but are not limited to: adrenal tumors such as adenoma, adrenal pheochromocytoma and adrenal ganglioneuroma; brain tumors such as meningioma and adenoma; peripheral nerve tumors such as neurofibroma and schwannoma; liver tumors such as adenoma; thyroid tumors such as follicular adenoma; parathyroid tumors such as adenoma; thymus tumors such as thymoma; salivary gland tumors such as pleomorphic adenoma; small intestine tumors such as villous adenoma; colon tumors such as tubulovillous adenoma, adenomatous polyp of colon, and polyposis coli; pancreas tumors such as serous cystadenoma; islet tumors such as pancreatic islet cell tumor; nasopharyngeal tumors such as nasal angiofibroma; ovarian tumors such as atypical proliferating mucinous neoplasm, Brenner tumor of ovary, mucinous cystadenoma, papillary cystadenoma, dermoid cyst of ovary, ovarian teratoma, ovarian fibroma, luteoma, and struma ovarii; uterine tumors such as uterine cellular leiomyoma and leiomyoma; placental tumors such as chorioangioma, partial hydatidiform mole, and complete hydatidiform mole; bone tumors such as cavernous hemangioma and giant cell tumor; soft tissue tumors such as cavernous hemangioma, desmoid tumor, lipoma, myelolipoma, and osteochondroma; joint tumors such as synovial chondromatosis; lung tumors such as carcinoid tumor, granular cell tumor, and hemangioma; myocardium tumors such as atrial myxoma; breast tumors such as fibroadenoma, intraductal papilloma and schwannoma; kidney tumors such as congenital mesoblastic nephroma; and skin tumors such as giant congenital intradermal nevus.

As used generally herein, the term “hyperproliferative disorders” refers to excess cell proliferation that is not governed by the usual limitation of normal growth. The term denotes malignant as well as nonmalignant cell populations. The excess cell proliferation can be determined by reference to the general population and/or by reference to a particular patient, e.g. at an earlier point in the patient's life. Hyperproliferative cell disorders can occur in different types of animals and in humans, and produce different physical manifestations depending upon the affected cells.

Hyperproliferative cell disorders include tumors as well as non-tumor conditions. A “tumor” here refers to an abnormal mass of tissue that results from excessive cell division that is uncontrolled and progressive, also called a neoplasm.

Examples of tumors include a variety of solid tumors such as laryngeal tumors, brain tumors, other tumors of the head and neck; colon, rectal and prostate tumors; breast and thoracic solid tumors; ovarian and uterine tumors; tumors of the esophagus, stomach, pancreas, and liver; bladder and gall bladder tumors; skin tumors such as melanomas and the like; and a fluid tumor such as leukemia.

A “solid tumor,” as used herein, refers to an abnormal mass of tissue that usually does not contain cysts or liquid areas. Solid tumors may be benign (not cancerous) or malignant (cancerous). Solid tumors have a distinct structure that mimics that of normal tissues and comprises two distinct but interdependent compartments: the parenchyma (neoplastic cells) and the stroma that the neoplastic cells induce and in which they are dispersed. Different types of solid tumors are named for the type of cells that form them. Examples of solid tumors are sarcomas, carcinomas, and lymphomas. Solid tumors are loci of tumor cells in which the majority of cells are tumor cells or tumor-associated cells.

More particularly, “tumor” as used herein refers to either benign (non-cancerous) or malignant tumors.

Malignant tumors include, but are not necessarily limited to: (A) breast cancer, including: (1) ductal carcinoma, including ductal carcinoma in situ (DCIS) (comedocarcinoma, cribriform, papillary, micropapillary), infiltrating ductal carcinoma (IDC), tubular carcinoma, mucinous (colloid) carcinoma, papillary carcinoma, metaplastic carcinoma, and inflammatory carcinoma; (2) lobular carcinoma, including lobular carcinoma in situ (LCIS) and invasive lobular carcinoma; and (3) Paget's disease of the nipple; (B) cancers of the female reproductive system, including: (1) cancers of the cervix uteri, including cervical intraepithelial neoplasia (Grade I), cervical intraepithelial neoplasia (Grade II), cervical intraepithelial neoplasia (Grade III) (squamous cell carcinoma in situ), keratinizing squamous cell carcinoma, nonkeratinizing squamous cell carcinoma, verrucous carcinoma, adenocarcinoma in situ, adenocarcinoma in situ, endocervical type, endometrioid adenocarcinoma, clear cell adenocarcinoma, adenosquamous carcinoma, adenoid cystic carcinoma, small cell carcinoma, and undifferentiated carcinoma; (2) cancers of the corpus uteri, including endometrioid carcinoma, adenocarcinoma, adenocanthoma (adenocarcinoma with squamous metaplasia), adenosquamous carcinoma (mixed adenocarcinoma and squamous cell carcinoma, mucinous adenocarcinoma, serous adenocarcinoma, clear cell adenocarcinoma, squamous cell adenocarcinoma, and undifferentiated adenocarcinoma; (3) cancers of the ovary, including serous cystadenoma. serous cystadenocarcinoma, mucinous cystadenoma, mucinous cystadenocarcinoma, endometrioid tumor, endometrioid adenocarcinoma, clear cell tumor, clear cell cystadenocarcinoma, and unclassified tumor; (4) cancers of the vagina, including squamous cell carcinoma and adenocarcinoma; and (5) cancers of the vulva, including vulvar intraepithelial neoplasia (Grade I), vulvar intraepithelial neoplasia (Grade II), vulvar intraepithelial neoplasia (Grade III) (squamous cell carcinoma in situ); squamous cell carcinoma, verrucous carcinoma, Paget's disease of the vulva, adenocarcinoma (NOS), basal cell carcinoma (NOS), and Bartholin's gland carcinoma; (C) cancers of the male reproductive system, including: (1) cancers of the penis, including squamous cell carcinoma; (2) cancers of the prostate, including adenocarcinoma, sarcoma, and transitional cell carcinoma of the prostate; (3) cancers of the testis, including seminomatous tumor, nonseminomatous tumor, teratoma, embryonal carcinoma, yolk sac tumor, and choriocarcinoma; (D) cancers of the cardiac system, including sarcoma (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma, and squamous cell carcinoma of the pharynx; (F) cancers of the lung, including squamous cell carcinoma (epidermoid carcinoma), variants of squamous cell carcinoma, spindle cell carcinoma, small cell carcinoma, carcinoma of other cells, carcinoma of intermediate cell type, combined oat cell carcinoma, adenocarcinoma, acinar adenocarcinoma, papillary adenocarcinoma, bronchiolo-alveolar carcinoma, solid carcinoma with mucus formation, large cell carcinoma, giant cell carcinoma, clear cell carcinoma, and sarcoma; (G) cancers of the gastrointestinal tract, including: (1) cancers of the ampulla of Vater, including primary adenocarcinoma, carcinoid tumor, and lymphoma; (2) cancers of the anal canal, including adenocarcinoma, squamous cell carcinoma, and melanoma; (3) cancers of the extrahepatic bile ducts, including carcinoma in situ, adenocarcinoma, papillary adenocarcinoma, adenocarcinoma, intestinal type, mucinous adenocarcinoma, clear cell adenocarcinom, segnet-ring cell carcinoma, adenosquamous carcinoma, squamous cell carcinoma, small cell (oat) carcinoma, undifferentiated carcinoma, carcinoma (NOS), sarcoma, and carcinoid tumor; (4) cancers of the colon and rectum, including adenocarcinoma in situ, adenocarcinoma, mucinous adenocarcinoma (colloid type; greater than 50% mucinous carcinoma), signet ring cell carcinoma (greater than 50% signet ring cell), squamous cell (epidermoid) carcinoma, adenosquamous carcinoma, small cell (oat cell) carcinoma, undifferentiated carcinoma, carcinoma (NOS), sarcoma, lymphoma, and carcinoid tumor; (5) cancers of the esophagus, including squamous cell carcinoma, adenocarcinoma, leiomyosarcoma, and lymphoma; (6) cancers of the gallbladder, including adenocarcinoma, adenocarcinoma, intestinal type, adenosquamous carcinoma, carcinoma in situ, carcinoma (NOS), clear cell adenocarcinoma, mucinous adenocarcinoma, papillary adenocarcinoma, signet-ring cell carcinoma, small cell (oat cell) carcinoma, squamous cell carcinoma, and undifferentiated carcinoma; (7) cancers of the lip and oral cavity, including squamous cell carcinoma; (8) cancers of the liver, including hepatoma (hepatocellular carcinoma), cholangiocarcinoma, hepatoblastoma, angiosarcoma, hepatocellular adenoma, and hemangioma; (9) cancers of the exocrine pancreas, including duct cell carcinoma, pleomorphic giant cell carcinoma, giant cell carcinoma, osteoclastoid type, adenocarcinoma, adenosquamous carcinoma, mucinous (colloid) carcinoma, cystadenocarcinoma, acinar cell carcinoma, papillary carcinoma, small cell (oat cell) carcinoma, mixed cell typed, carcinoma (NOS), undifferentiated carcinoma, endocrine cell tumors arising in the islets of langerhans, and carcinoid; (10) cancers of the salivary glands, including acinic (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well differentiated or low grade), and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenosquamous carcinoma, squamous cell carcinoma, small cell carcinoma, undifferentiated carcinoma, lymphoma, sarcoma, and carcinoid tumor; and (12) cancers of the small intestine, including adenocarcinoma, lymphoma, carcinoid tumors, Kaposi's sarcoma, leiomyoma, hemangioma, lipoma, neurofibroma, and fibroma; (H) cancers of the urinary system, including: (1) cancers of the kidney, including renal cell carcinoma, carcinoma of Bellini's collecting ducts, adenocarcinoma, papillary carcinoma, tubular carcinoma, granular cell carcinoma, clear cell carcinoma (hypernephroma), sarcoma of the kidney, and nephroblastoma; (2) cancers of the renal pelvis and ureter, including transitional cell carcinoma, papillary transitional cell carcinoma, squamous cell carcinoma, and adenocarcinoma; (3) cancers of the urethra, including transitional cell carcinoma, squamous cell carcinoma, and adenocarcinoma; and (4) cancers of the urinary bladder, including carcinoma in situ, transitional urothelial cell carcinoma, papillary transitional cell carcinoma, squamous cell carcinoma, adenocarcinoma, undifferentiated; (I) cancers of muscle, bone, and soft tissue, including: (1) cancers of bone, including: (a) bone-forming: osteosarcoma; (b) cartilage-forming: chondrosarcoma and mesenchymal chondrosarcoma; (c) diant cell tumor, malignant; (d) Ewing's sarcoma; (e) vascular tumors: hemangioendothelioma, hemangiopericytoma, and angiosarcoma; (f) connective tissue tumors: fibrosarcoma, liposarcoma, malignant mesenchymoma, and undifferentiated sarcoma; and (g) other tumors: chordoma and adamantinoma of long bones; (2) cancers of soft tissues, including: alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, rhabdomyosarcoma, synovial sarcoma, and sarcoma (NOS); (3) cancers of the nervous system, including cancers of the skull (osteoma, hemangioma, granuloma, xanthoma, osteitis deformans), cancers of the meninges (meningioma, meningiosarcoma, gliomatosis), cancers of the brain (astrocytoma, medulloblastoma, glioma, ependymoma, germinoma (pilealoma), glioblastoma multiform, oligodendroglioma, schwannoma, retinoblastoma, congenital tumors), and cancers of the spinal cord neurofibroma, meningioma, glioma, sarcoma); (4) hematologic cancers, including myeloid leukemia (acute and chronic), acute lymphloblastic leukemia, chronic lymphocytic leukemia, myeloproliferative diseases, multiple myeloma; myelodysplastic syndrome), Hodgkin's disease, and non-Hodgkin's lymphoma (malignant lymphoma); (5) cancers of the endocrine system, including: (a) cancers of the thyroid gland, including papillary carcinoma (including those with follicular foci), follicular carcinoma, medullary carcinoma, and undifferentiated (anaplastic) carcinoma; and (b) neuroblastomas, including sympathicoblastoma, sympathicogonioma, malignant ganglioneuroma, gangliosympathicoblastoma, and ganglioneuroma; (6) cancers of the skin, including squamous cell carcinoma, spindle cell variant of squamous cell carcinoma, basal cell carcinoma, adenocarcinoma developing from sweat or sebaceous gland, and malignant melanoma; (7) cancers of the eye, including: (a) cancers of the conjunctiva, including carcinoma of the conjunctiva; (b) cancers of the eyelid, including basal cell carcinoma, squamous cell carcinoma, melanoma of the eyelid, and sebaceous cell carcinoma; (c) cancers of the lacrimal gland, including adenocarcinoma, adenoid cystic carcinoma, carcinoma in pleomorphic adenoma, mucoepidermoid carcinoma, and squamous cell carcinoma; (d) cancers of the uvea, including spindle cell melanoma, mixed cell melanoma, and epithelioid cell melanoma; (e) cancers of the orbit, including sarcoma of the orbit, soft tissue tumor, and sarcoma of bone; and (f) retinoblastoma.

Examples of nontumor hyperproliferative disorders include but are not limited to myelodysplastic disorders; cervical carcinoma-in-situ; familial intestinal polyposes such as Gardner syndrome; oral leukoplakias; histiocytoses; keloids; hemangiomas; inflammatory arthritis; hyperkeratoses and papulosquamous eruptions including arthritis-related eruptions. Also included are viral induced hyperproliferative diseases such as warts and EBV induced disease (i.e., infectious mononucleosis), scar formation, blood vessel proliferative disorders such as restenosis, atherosclerosis, in-stent stenosis, vascular graft restenosis, etc.; fibrotic disorders; psoriasis; glomerular nephritis; macular degenerative disorders; benign growth disorders such as prostate enlargement and lipomas; autoimmune disorders and the like.

Compositions according to the present invention can also be administered for the treatment of cardiac dysrhythmias, including but not limited to the Wolff-Parkinson-White syndrome and atrioventricular nodal reentrant tachycardia ventricular tachycardia (VT), atrial tachycardias, atrial flutter and atrial fibrillation supraventricular tachycardias.

Compositions according to the present invention can also be administered for the treatment of endometriosis, uterine fibroid (uterine leiomyomata) menorrhagia, cervical erosion, cervical polyp, and related conditions.

Compositions according to the present invention can also be administered for the treatment of the defects or disorders of intervertebral discs including but not limited to annular fissures, fragmentation of the nucleus pulposus, contained herniation (a herniated intervertebral disc), and degenerative intervertebral discs.

Compositions according to the present invention can also be administered for the treatment of additional diseases or conditions, including, but not limited to, Alzheimer's disease, osteoporosis, sleep apnea, erectile dysfunction, McArdle disease, and carbohydrate metabolism disorders.

Compositions according to the present invention can also be administered for reducing aging or fatigue. As used herein, the term “reducing aging” refers to lessening, ameliorating, or relieving the deleterious effects of aging (e.g., low vigor, memory loss, weakened vision or hearing, and joint pain) in a subject. As used herein, the term “reducing fatigue” refers to lessening, ameliorating, or relieving one or more of the symptoms of fatigue (low energy, poor endurance, and attention deficits) in a subject.

The subject to be treated can be a human patient or a socially or economically important animal, including, but not limited to, a dog, a cat, a horse, a cow, a goat, a sheep, or a pig. Compositions according to the present invention can be formulated for treatment of non-human mammalian species such as, but not limited to, those described above and can be used in veterinary medicine. Methods according to the present invention are not limited to the treatment of humans and can be adapted for use in veterinary medicine.

The composition described above can be in dry form (e.g., powder or tablet) or in aqueous form (e.g., beverage or syrup). It can be a dietary supplement or a pharmaceutical formulation (containing a pharmaceutically acceptable carrier). It can also be a drink or a food product. Examples include tea (e.g., a tea drink and the contents of a tea bag), soft drinks, juice (e.g., a fruit extract and a juice drink), milk, coffee, cookies, cereals, chocolates, and snack bars.

The first and second agents described above include active compounds, as well as their salts, prodrugs, and solvates, if applicable. A salt, for example, can be formed between an anion and a positively charged group (e.g., amino) on an agent. Suitable anions include chloride, bromide, iodide, sulfate, nitrate, phosphate, citrate, methanesulfonate, trifluoroacetate, acetate, chlorophenyoxyacetate, malate, tosylate, tartrate, fumarate, glutamate, glucuronate, lactate, glutarate, benzoate, embonate, glycolate, pamoate, aspartate, parachlorophenoxyisobutyrate, formate, succinate, cyclohexanecarboxylate, hexanoate, octanoate, decanoate, hexadecanoate, octadecanoate, benzenesulphonate, trimethoxybenzoate, paratoluenesulphonate, adamantanecarboxylate, glycoxylate, pyrrolidonecarboxylate, naphthalenesulphonate, 1-glucosephosphate, sulfite, dithionate, and maleate. Likewise, a salt can also be formed between a cation and a negatively charged group (e.g., carboxylate) on an agent. Suitable cations include sodium ion, potassium ion, magnesium ion, calcium ion, and an ammonium cation such as tetramethylammonium ion. The agents also include salts containing quaternary nitrogen atoms. Examples of prodrugs include esters and other pharmaceutically acceptable derivatives, which, upon administration to a subject, are capable of providing active compounds. A solvate refers to a complex formed between an active compound and a pharmaceutically acceptable solvent. Examples of pharmaceutically acceptable solvents include water, ethanol, isopropanol, ethyl acetate, acetic acid, and ethanolamine.

In some alternatives, the composition can include one or more additional active ingredients unless such additional active ingredients are excluded by a definition of the composition that includes the phrase “consisting essentially of.”

The invention is illustrated by the following Examples. These Examples are included for illustrative purposes only, and is not intended to limit the invention.

### EXAMPLE 1

### Solubilization Test of Poloxamer 188/407 to Aspirin in Ethanol

Methods

(1) 0%,10%, 20%, 30%, 40% (m/v) of Poloxamer 188/407 anhydrous ethanol solution were prepared respectively,10 ml of each solution was added into five sealed centrifuge tubes respectively (each centrifuge tube was numbered accordingly).

(2) Each centrifuge tube was added with adequate amount of Aspirin based on the pre-test data (please refer to Table 1: 0 h data), and shaken to dissolution at room temperature (30° C.).

(3) The tubes were observed every certain time interval, as shown in Table 1. 0.1 g of Aspirin would be added into the tube when complete dissolution was observed, then sealed and continued shaking. In cases which the drug was not dissolved, the previous amount of drug dissolved would be recorded as the maximum amount needed for dissolution.

(4) Dissolution observation was continued until some amount of drug compound could not be dissolved.

(5) The observation of each tube was recorded in Table 1

TABLE 1
Observation Records

| Poloxamer | | | | | | | | | | | | | |
|---|---|---|---|---|---|---|---|---|---|---|---|---|---|
| 188/407 | Time Point and Amount of Aspirin Added | | | | | | | | | | | | |
| Quantity | 0 h | 0.5 h | 1 h | 1.5 h | 2 h | 4 h | 5 h | 6 h | 7 h | 8 h | 24 h | 32 h | 36 h |
| 0% | 2.00 | | | | | | | | | | | | |
| 10% | 2.80 | | | | | | | | | | | | |
| 20% | 2.90 | | | | | | | | | | | | |
| 30% | 3.00 | | | | | | | | | | | | |
| 40% | 3.00 | | | | | | | | | | | | |

Results

Poloxamer 188 and 407 anhydrous ethanol increase the solubility of Aspirin, in which the solubility increases with Poloxamer concentration. However, the degree of solubilization is disproportional to the concentration of Poloxamer, in which the degree of solubilization decreased with increased Poloxamer concentration. The results are shown in Tables 2 and 3.

TABLE 2
Solubilization Effect of Poloxamer 188 to Aspirin

| | Amount | |
|---|---|---|
| Poloxamer 188 | Dissolved | Solubilization |
| Quantity (%) | (g) | Rate (%) |
| 0 | 2.50 | 0 |
| 10 | 2.80 | 12 |
| 20 | 3.00 | 20 |
| 30 | 3.10 | 24 |
| 40 | 3.20 | 28 |

TABLE 3
Solubilization Effect of Poloxamer 407 to Aspirin

| Poloxamer 407 | | |
|---|---|---|
| Quantity (%) | Amount Dissolved (g) | Solubilization Rate (%) |
| 0 | 2.50 | 0 |
| 10 | 2.80 | 12 |
| 20 | 3.00 | 20 |
| 30 | 3.10 | 24 |
| 40 | 3.20 | 28 |

### EXAMPLE 2

### Gelation Temperature Measurement Test

Methods

(1) 10 mL of temperature-sensitive formulation mixture was prepared. The formulation includes equal volumes of Mixture A and Mixture B, below.

Mixture A, per each 1-mL aliquot, contains: 75 mg metformin hydrochloride; 5 mg serotonin creatinine sulfate complex; 68.75 mg poloxamer 407; 18.75 mg poloxamer 188; 0.5 mg sodium metabisulfite; and water for injection to a final volume of 1 mL. Mixture B, per each 1-mL aliquot, contains: 200 mg aspirin; 450 mg poloxamer 407; 5 mg tartaric acid; and anhydrous ethanol to a final volume of 1 mL.

(2) The drug mixture was then added into a 25-mL serum vial (containing stirrer).

(3) The serum vial was placed in a 28° C. water bath for 15 minutes.

(4) When stirring was started, the stirrer was observed whether it is rotatable. The previous temperature degree of gel was recorded as the gelation temperature when the stirrer stops rotating. On the other hand, if the stirrer was still rotating, the water bath temperature would be adjusted based on the table below and placed the serum vial in the water bath for another 15 minutes and the rotation of stirrer was then observed again.

(5) The water bath temperature was increased until the stirrer was not rotating; the gel temperature was then recorded.

(6) Each of the observed condition was then recorded to the following table (Table 4).

TABLE 4
Observation Record

| | Water Bath Temperature and Gelation Observation | | | | | | | |
|---|---|---|---|---|---|---|---|---|
| | | | | | | 36° | | 38° |
| Vial | 28° C. | 30° C. | 32° C. | 34° C. | 35° C. | C. | 37° C. | C. |
| 1 | | | | | | | | |
| 2 | | | | | | | | |
| 3 | | | | | | | | |

Result: The gelation temperature of the formulation mixture is around 35-36° C. This is shown in Table 5.

TABLE 5
Results

| | Water Bath Temperature and Gelation Observation | | | | | | | |
|---|---|---|---|---|---|---|---|---|
| | | | | | | 36° | | 38° |
| Vial | 28° C. | 30° C. | 32° C. | 34° C. | 35° C. | C. | 37° C. | C. |
| 1 | − | − | − | − | + | | | |
| 2 | − | − | − | − | + | | | |
| 3 | − | − | − | − | − | + | | |

### EXAMPLE 3

### Dissolution Release Test

Methods

(1) 10 mL of formulation solution was prepared. This formulation solution was prepared according to the following instructions: A 10-mL solution was prepared comprising equal volumes of Mixture A and Mixture B. Mixture A comprised, per 1-mL aliquot: 75 mg of metformin hydrochloride; 5 mg of serotonin creatinine sulfate complex; 68.75 mg of poloxamer 407; 18.75 mg of poloxamer 188; 0.5 mg sodium metabisulfite; and water for injection to a total volume of 1 mL. Mixture B comprised, per 1-mL aliquot: 200 mg of aspirin, 450 mg of poloxamer 407; 5 mg of tartaric acid; and anhydrous alcohol to a total volume of 1 mL.

(2) The solution was then slowly added into 15-mL centrifuge tubes (each tube 3 mL) by ensuring that no solution stuck on the test tube wall while keeping the liquid level even.

(3) The test tube was placed in the cell incubator for 30 minutes at 37° C.

(4) 3 mL of DMEM (red) medium at 37° C. was added while waiting for the mixture to transform to the gel form (colorless).

(5) The changes between the red and colorless interface were continuously observed and recorded.

(6) The observations for each tube were recorded in Table 6.

TABLE 6

| | Observation Time Point and the Amount of Gel Remain | | | | | | | | | | | | |
|---|---|---|---|---|---|---|---|---|---|---|---|---|---|
| Tube No. | 0 h | 0.5 h | 1 h | 1.5 h | 2 h | 2.5 h | 3 h | 3.5 h | 4 h | 4.5 h | 5 h | 5.5 h | 6 h |
| 1 | 3.0 | | | | | | | | | | | | |
| 2 | 3.0 | | | | | | | | | | | | |
| 3 | 3.0 | | | | | | | | | | | | |

The results show that when exposing the drug mixture to culture medium, the dissolution process slows down. There is a positive correlation between the dissolution quantity and the amount of time. Based on the linear regression equation estimation, the time needed for the formulation to completely dissolve is around 14.2-20.9 hours under body temperature, demonstrating an obvious sustained-release effect.

Results from Test 1 are shown in Table 7 and results from Test 2 are shown in Table 8.

TABLE 7

| Test | | | | | | | | | | | | | |
|---|---|---|---|---|---|---|---|---|---|---|---|---|---|
| Tube | Observation Time Point and the Amount of Gel Remain | | | | | | | | | | | | |
| No. | 0 h | 0.5 h | 1 h | 1.5 h | 2 h | 2.5 h | 3 h | 3.5 h | 4 h | 4.5 h | 5 h | 5.5 h | 6 h |
| b10-1 | 3.00 | 2.80 | 2.75 | 2.70 | 2.60 | 2.50 | 2.40 | 2.30 | 2.20 | 2.10 | 2.00 | 1.90 | 1.75 |
| b10-2 | 3.00 | 2.75 | 2.70 | 2.60 | 2.50 | 2.40 | 2.30 | 2.20 | 2.10 | 2.00 | 1.90 | 1.80 | 1.65 |
| b10-3 | 3.00 | 2.75 | 2.70 | 2.60 | 2.50 | 2.40 | 2.30 | 2.20 | 2.10 | 2.00 | 1.85 | 1.75 | 1.55 |
| Average | 3.00 | 2.77 | 2.72 | 2.63 | 2.53 | 2.43 | 2.33 | 2.23 | 2.13 | 2.03 | 1.92 | 1.82 | 1.65 |

TABLE 8

| Test | | | | | | | | | | | | | |
|---|---|---|---|---|---|---|---|---|---|---|---|---|---|
| Tube | Observation Time Point and the Amount of Gel Remain | | | | | | | | | | | | |
| No. | 0 h | 0.5 h | 1 h | 1.5 h | 2 h | 2.5 h | 3 h | 3.5 h | 4 h | 4.5 h | 5 h | 5.5 h | 6 h |
| b10-1 | 3.00 | 3.00 | 2.90 | 2.80 | 2.70 | 2.60 | 2.50 | | 2.50 | | 2.40 | | 2.25 |
| b10-2 | 3.00 | 3.00 | 2.90 | 2.80 | 2.70 | 2.50 | 2.45 | | 2.40 | | 2.25 | | 2.10 |
| Average | 3.00 | 3.00 | 2.90 | 2.80 | 2.70 | 2.55 | 2.48 | | 2.45 | | 2.33 | | 2.18 |

### Advantages of the Invention

Compositions and methods according to the present invention are effective in treating a number of diseases and conditions, including metabolic syndrome and diseases and conditions associated with metabolic syndrome, hyperproliferative diseases including cancer, AIDS, Parkinson's disease, polycystic ovarian syndrome, Alzheimer's disease, osteoporosis, sleep apnea, erectile dysfunction, McArdle disease, and carbohydrate metabolism disorders, cardiac dysrhythmias; endometriosis, uterine fibroid (uterine leiomyomata) menorrhagia, cervical erosion, cervical polyp, and related conditions, defects or disorders of intervertebral discs. Compositions and methods according to the present invention are well tolerated, produce few if any side effects, and can be used together with other known pharmaceutically active compounds and compositions for treating these conditions. The sustained-release properties of the present invention improve bioavailability and provide improved pharmacokinetic properties and avoid the possibility of overdosage.

Compositions and methods according to the present invention possess industrial applicability as compositions and methods for the preparation of a medicament to treat the diseases and conditions described above.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed herein:

1. A pharmaceutical formulation comprising three active pharmaceutical ingredients, a solvent, and at least one non-ionic surfactant.

2. The formulation of claim 1, wherein the three active pharmaceutical ingredients are Metformin, Aspirin, Serotonin or the salts and/or hydrates thereof.

3. The formulation of claim 1, wherein the non-ionic surfactant is a α-hydro-ω-hydroxypoly(oxyethylene)ₐ-poly(oxypropylene)_{b}-poly(oxyethylene)_{b} block copolymer.

4. The formulation of claim 1, wherein the non-ionic surfactant is poloxamer 407 or poloxamer 188.

5. The formulation of claim 1, wherein the non-ionic surfactant functions as an absorption enhancer.

6. The formulation of claim 1, wherein the non-ionic surfactant functions as an emulsifier, a solubilizer, or a stabilizer.

7. The formulation of claim 1, wherein the non-ionic surfactant functions as a temperature control agent or a slow-release agent.

8. The formulation of claim 4, wherein the poloxamer 407 and/or poloxamer 188 functions as a temperature control agent or a slow-release agent.

9. The formulation of claim 1 wherein the formulation comprises: (i) metformin; (ii) serotonin or the salt thereof and/or hydrate thereof; (iii) water; (iv) poloxamer 407; and (v) poloxamer 188.

10. The formulation of claim 1, wherein the formulation comprises: (i) metformin; (ii) aspirin; (iii) ethanol; and (iv) poloxamer 407.

11. The formulation of any one of claims 1-10, wherein the formulation is an injectable formulation or an oral dosage formulation.
